(19) United States
(12) Patent Application Publication
(10) Pub. No.: US 2004/0267082 A1
(43) Pub. Date: Dec. 30, 2004

(54) Device for treating headache

(76) Inventor: Lucio Brandoli, Castelnuovo Rangone (IT)

Correspondence Address: NIXON & VANDERHYE, PC, 1100 N GLEBE ROAD, 8TH FLOOR, ARLINGTON, VA, 22201-4714, US

(21) Appl. No.: 10/498,376

(22) PCT Filed: Dec. 9, 2002
(86) PCT No.: PCT/EP02/13947

(30) Foreign Application Priority Data
Dec. 10, 2001 (IT) MO2001A000245

(51) Int. Cl. A61N 2/00; A61B 19/00

(52) U.S. Cl. 600/9; 128/897

(57) ABSTRACT

An apparatus for treating headache comprises a body so shaped as to be received in the mouth of a patient, at least a part of said body being made of metallic material, a method for treating headache comprising introducing a body made of metallic material in the mouth of a patient.

The invention relates to a device for treating migraine and headache in general.

Migraine is characterized by recurrent attacks of headache of strong intensity, which generally affect only one half of the head and last from some hours to some days.

Migraine is a somewhat common pathology: actually, as results from some researches which have been carried out, about 15% of the female population and about 6% of the male population suffer from said pathology.

Said pathology not only causes severe pains, but is also quite disabling since it prevents the suffering person from executing, during the attacks, the normal activities, for example the working activities, especially in the case that said activities require particular attention and concentration.

Other, rather common pathologies are further known, such as the so-called “tension headache” and “cluster headache”, that, even though partially differing from the migraine, appear nevertheless with symptoms that comprise sharp pains at the head.

For treating migraine and headaches in general, analgesic medicines are known that reduce, and preferably remove, the pain at the head.

A drawback of such analgesics is that they can result ineffective in the case of headache attacks of particular intensity.

Drugs of various typologies are further known that oppose against the symptoms of migraine by promoting an action for reducing the diameter of the blood vessels of the skull.

A general drawback of the anti-migrainous drugs is that they are badly tolerated by the organism of the patient.

Since such drugs are usually distributed in the form of pills, capsules, and/or tablets, a protracted assumption can damage the digestive system of a patient, in particular the stomach, as well as to produce intoxication and consequent hepatic problems.

A further drawback of said drugs is that they can produce collateral highly troublesome effects for the patient and, in certain cases, even seriously compromise health thereof.

A still further drawback is that said drugs can not be used by any patient: actually, patients that already undergo a therapy based on medicines incompatible with said drugs can not use the above mentioned drugs freely.

In addition, women that are in pregnancy condition or during nursing are strongly recommended not to use such drugs.

An object of the present invention is to improve the known systems for treating migraines.

Another object is to obtain a device for treating migraines that can be used by any type of person.

A further object is to provide a device for treating migraines, which does not cause undesired collateral effects when used.

In a first aspect of the invention, a device for treating headache is provided comprising a body so shaped as to be received in the mouth of a patient, characterized in that at least part of said body is made of metallic material.

Although the device may have any shape, in a preferred version, the body comprises a first portion suitable for being received in the mouth of the patient and a further portion protruding from the mouth of the patient.

Advantageously, since the body has a quite limited size, a user can keep said body inside the mouth by simply tightening said body between the lips and/or the teeth.

Therefore, a patient can use the device according to the invention during any activity.

In particular, since the hands of the patient are free, the device according to the invention may be used during any working activity.

In a further preferred version, the first portion is made with said metallic material.

In a further advantageous version, the second portion comprises a safety element arranged for preventing the body from being accidentally ingested by a user.

In a still further preferred version, the body comprises a nail provided with a head suitable for being introduced into the mouth of the patient and tip suitable for protruding from the above-mentioned mouth.

Tests carried out on a plurality of patients have allowed verifying that the pain caused by acute attacks of migraine is substantially removed when a patient places into his mouth a farrier nail made of soft iron.

Such nail is placed in the mouth as soon as the symptoms of the migraine appear and is kept partially inserted inside the oral cavity until said symptoms disappear.

The performed tests have enabled to verify that the device according to the invention leads to the above mentioned effects when kept inside the mouth of the patient for an interval of time extending from a few minutes to some hours, up to a maximum of four hours.

The researches that have been carried out allow to attest that a high number of metals result suitable for being used for manufacturing the device according to the invention.

Said material preferably comprises, iron (in particular soft iron), copper (in particular copper through which electric current has been caused to pass) or any other type of magnetic material.

In a second aspect of the invention, there is provided the use of a metallic body so shaped as to be received in the mouth of a patient as a device for treating headache.

Owing to these aspects of the invention, it is possible to obtain a device for treating migraine that can be used by patients of any age, sex and health condition, without contra-indications.

In addition, such device can be used also by patients suffering from pathologies involving pharmacological treatments, since the use of the device according to the invention is not in conflict with the medicines required by said treatments.

In addition, the device according to the invention may be simply used also during any type of activity, without limiting the possibilities of action of the user.

Furthermore, the device disclosed in the present invention is quite inexpensive and not very susceptible to wear: therefore said device can be effectively used for a high number of times.

The invention may be better understood and carried out with reference to the accompanying drawings, which show some exemplifying and not limiting embodiments thereof, in which:

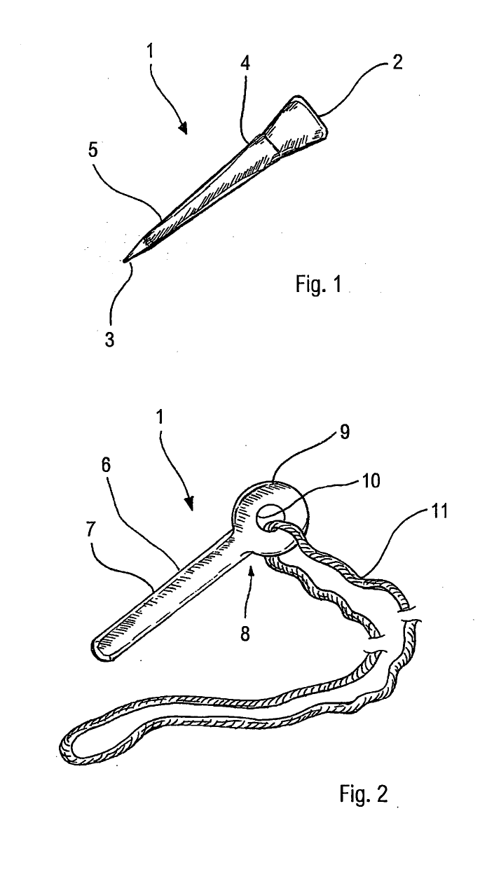

FIG. 1 is a schematic perspective view of a device according to the invention;

FIG. 2 is a view like that of FIG. 1 showing a preferred version of the device according to the invention.

FIG. 1 shows a device for treating headache 1 comprising a farrier nail, said farrier nail being preferably made of soft iron.

The device 1 is provided with a head 2 suitable for being received in the mouth of a patient together with a first stem portion 3 of the nail, said first stem portion 3 being arranged near to the head 2.

The device 1 is further provided with a tip 3 and a second stem portion 5, arranged close to the tip 3, which are suitable for projecting from the mouth of the patient.

It has been observed that, since the device 1 comprises the tip 3, patients are caused to bear in mind that a sharp object is placed inside their mouth and therefore the above-mentioned patients pay more attention not to accidentally ingest the device itself.

FIG. 2 shows a device 1 according to the invention comprising a body 6 suitable for being received in the mouth of a patient.

The body 6 comprises a metallic elongated element 7 having an end 8 to which a ring 9 is connected.

The ring 9 has shape and size such to prevent the device 1 to be completely inserted into the mouth.

The ring 9 comprises a central hole 10 suitable for allowing a cord 11 to be connected to the ring 9, said cord 11 preventing the device 1 from being ingested by the patient.

The ring 9 may be made of any material and preferably is obtained with the metallic material with which the elongated element 7 is made.

1-15. (canceled)

16. Apparatus for treating headache, comprising a body so shaped as to be received in the mouth of a patient, wherein at least part of said body is made of metallic material.

17. Apparatus according to claim 16, wherein said body comprises a first portion suitable for being received in said mouth and a further portion protruding from said mouth.

18. Apparatus according to claim 17, wherein at least said first portion comprises said metallic material.

19. Apparatus according to claim 17, wherein said second portion comprises a safety element having shape and size such to prevent said patient from accidentally ingesting said body.

20. Apparatus according to claim 16 or 17, wherein said body comprises a nail.

21. Apparatus according to claim 20, wherein said nail is a farrier-type nail.

22. Apparatus according to claim 20 as appended to claim 17, wherein said first portion comprises a head and a stem portion of said nail and said second portion comprises a tip and a remaining stem portion of said nail.

23. Apparatus according to claim 19, wherein said safety element comprises ring element provided with hole element.

24. Apparatus according to claim 23, wherein said hole element is suitable for receiving safety cord device arranged for preventing said apparatus from being ingested by a patient.

25. Apparatus according to claim 16, wherein said metallic material comprises iron.

26. Apparatus according to claim 25, wherein said iron comprises soft iron.

27. Apparatus according to claim 16, wherein said metallic material comprises copper.

28. Apparatus according to claim 27, wherein said copper comprises copper through which electric current has been caused to pass.

29. Apparatus according to claim 16, wherein said metallic material comprises a magnetic material.

30. Use of a metallic body shaped so as to be received in the mouth of a patient as apparatus for treating headache.

31. Method for treating headache, comprising introducing a body made of metallic material in the mouth of a patient.

32. Method according to claim 31, wherein said introducing comprises inserting a first portion of said body so that a further portion of said body protrudes from said mouth.

33. Method according to claim 32 and further comprising providing said further portion with a safety element so as to prevent said patient from accidentally ingesting said body.

34. Method according to claim 33, wherein said providing comprises obtaining a ring element provided with hole in said further portion.

35. Method according to claim 34, and further comprising placing a cord device into said hole for preventing said body from being ingested by a patient.

36. Method according to claim 31 and further comprising treating said metallic material by causing electric current to pass through said metallic material.
